(19) United States
(12) Patent Application Publication
(10) Pub. No.: US 2024/0237958 A1
(43) Pub. Date: Jul. 18, 2024

(54) IMAGING DEVICE AND IMAGING METHOD

(71) Applicant: Planmed Oy, Helsinki (FI)

(72) Inventor: Leo ASPELUND, Helsinki (FI)

(21) Appl. No.: 18/563,130

(22) PCT Filed: May 24, 2022
(86) PCT No.: PCT/FI2022/050361

§ 371 Date: Nov. 21, 2023

(30) Foreign Application Priority Data
May 24, 2021 (FI) 20215612
May 24, 2021 (FI) 20215613

(51) Int. Cl. A61B 6/00; A61B 6/02; A61B 6/03; A61B 6/04; A61B 6/50

(52) U.S. Cl. CPC A61B 6/4417; A61B 6/027; A61B 6/035; A61B 6/0421; A61B 6/502; A61B 6/4441

(57) ABSTRACT

The invention relates to imaging devices and methods for x-ray imaging a breast. wherein when changing an operation mode of the imaging device for CT imaging a specific-kind breast positioning structure may be connected to the imaging device.

### TECHNICAL FIELD

The present disclosure relates generally to imaging devices, and more specifically to imaging devices and methods for x-ray imaging a breast of a patient.

### BACKGROUND

Mammography concerns detecting breast tumors and changes in breast tissue. Mammography is the most widely used method in the diagnosis of breast cancer. It combines good diagnostic accuracy, cost-effectiveness and its general applicability to different people. Mass screening of female population aims to detect cancer as early as possible. Early detection enables more efficient treatment of the disease. In mammography, typically a low-energy radiation x-ray device specifically designed for imaging breasts is used.

One typical kind mammography device comprises a vertical frame and a rotating C-arm connected thereto. The rotating C-arm is connected to the vertical frame via a rotating axis. The rotating axis allows the C-arm to be rotated to different orientations. The vertical frame may be vertically adjustable by means of motors so that the C-arm can be positioned for patients of different heights.

Breast compression may be used in connection with mammography imaging. Radiographing a thick object requires using higher energy radiation to enable sufficient penetration of X-ray quanta through the object. When the energy of the radiation increases, the contrast of the soft tissues in the image decreases. Because the breast is mainly fluid, squeezing it does not change its density but spreads the tissue over a wider area. Breast propagation in compression also reduces aggregation due to tissue overlap. Compression also reduces the radiation dose. A thick subject also scatters radiation more than a thin subject, which reduces the sharpness of the image. Compression can also reduce blurring of the image caused by motion during an exposure.

Compression is typically applied in traditional 2D mammography for projection-specific examination when the viewing direction and the direction of compression are parallel. Also, tomographic imaging has been applied in the context of breast radiography. By tomographic imaging one may be able to detect features within an object which in a mere conventional 2D image get imaged on top of each other and, thus, potentially remain at least partly undetected.

A method called tomosynthesis has been applied in the context of traditional mammography apparatus, wherein several images of a breast are taken over a limited tomographic angle while the breast remains in the same position. In tomosynthesis, 2D images are taken typically from different directions covering an angle range of +/−10 degrees or something of that order.

Traditional mammography apparatus typically is not configured to enable an actual computed tomography (CT), like cone beam computed tomography (CBCT). Prior art apparatus designed for breast CT include ones in which a breast of a patient, who is in a prone position, hangs through an opening of a horizontally extending patient support table.

### SUMMARY

An object of the present disclosure is to provide a solution that overcomes at least partially the problems encountered in prior art and provides an innovative solution for versatile breast x-ray imaging. The object is achieved by an imaging device and method with their various embodiments as disclosed herein.

The present disclosure enables computed tomography in a typical traditional 2D mammography device. This is accomplished by arranging to be connectable to a conventional mammography apparatus a breast positioning structure specifically designed for breast computed tomography. In particular, cone-beam computed tomography (CBCT). The solution allows for a relative rotational movement of the breast positioning structure and the rotating C-arm. Thereby, the breast positioning structure may remain in place relative to a standing or a sitting patient during rotation of the rotating C-arm.

An example of advantages over the conventional mammography devices the current disclosure brings along is an ability to generate CT images of a breast while using a traditional-kind 2D mammography imaging device. This may enable better contrast separation for dense breast tissues. Other advantages include that in computed tomography mode, the patient may place a breast in the imaging volume of the imagine device by herself and the breast will not be compressed, so imaging is painless and the examination therefore more pleasant for the patient.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various arrangements without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, examples of constructions are shown in the drawings. However, the present disclosure is not limited to specific methods or structures disclosed herein.

Moreover, those in the art will understand that the drawings may not be to scale. Like elements may have been indicated by identical numbers.

Embodiments of the present disclosure will now be described, by way of example only, with reference to the following Figures wherein:

FIG. 1A is a perspective view of a breast positioning in accordance with an embodiment of the present structure disclosure;

FIG. 1B is a side view of a breast positioning structure in accordance with an embodiment of the present disclosure;

FIG. 2 is a side view of an imaging device in accordance with an embodiment of the present disclosure without a breast positioning structure;

FIG. 3 is a side view of an imaging device shown in FIG. 3 with a breast positioning structure;

FIG. 4 is an exploded perspective view of an imaging device shown in FIG. 3 in which the breast positioning structure and the imaging device separated.

FIG. 5A is an embodiment of the present disclosure in which a patient stands at an imaging device during imaging and the rotatable arm rotates around the patient's breast over a scanning angle of about 200 degrees;

FIG. 5B is an embodiment of the present disclosure in which a patient sits at an imaging device during imaging and a rotating arm rotates around the patient's breast over a scanning angle of about 200 degrees;

FIG. 5C is a side view of FIG. 5B, in which the patient sits at the imaging device during imaging and a rotating arm rotates about the patient's breast over a scanning angle of about 200 degrees.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF THE DRAWINGS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

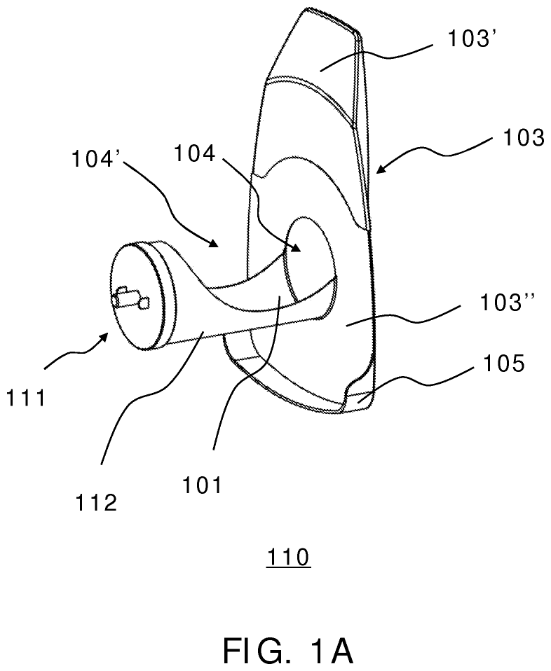

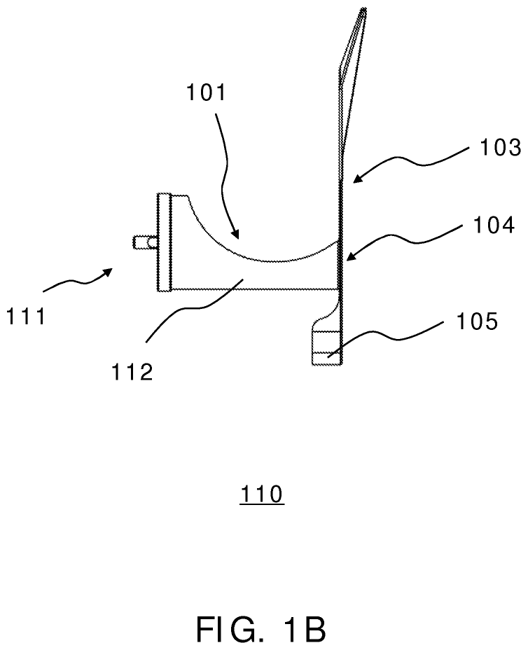

Referring to FIGS. 1A and 1B, a breast positioning structure 110 according to an embodiment of this disclosure is shown. FIG. 1A is a perspective view of the breast positioning structure 110 and FIG. 1B is a side view of the breast positioning structure 110. The breast positioning structure 110 comprises a first component in form of an elongated support element 112 extending in a first direction and having at its first end a connecting means 111. The breast positioning structure 110 further comprises at the second end of the elongated support element 112, as integrated thereto, a second component of the breast positioning structure 110, which second component is a plate-like protective cover 103. The plate-like protective cover 103 comprises a surface extending essentially on a plane in a second direction which is substantially at right angles to the first direction in which the elongated support element 112 extends.

The elongated support element 112 shown in FIG. 1A comprises a tubular but for a part of its length an open breast positioning surface 101. The plate-like protective cover 103 comprises an opening 104 which unites to the breast positioning surface 101 of the elongated support element 112.

The opening in the plate-like protective cover 103 uniting to inner surface of the elongated support element 112 can be referred to as a first opening 104 while, in case there is an open section in the elongated tubular support element 112, that can be referred to as a second opening 104′.

The breast positioning structure 110 may have a wall thickness of 2-5 mm, for example 2 mm.

The protective cover 103 has a lower portion 103″ configured to extend substantially at right angles to the elongated support element 112 and an upper portion 103′ at an angular orientation relative to the lower portion 103″, which upper portion 103′ is configured to extend in a direction away from the lower portion. The upper portion 103′ is shaped to taper upwards. The lower portion 103″ is wider than the upper portion 103′. In connection with the lower portion 103″ there is a protection part 105 which extends from the plate-like protection cover 103 in the same direction as the elongated support element 112.

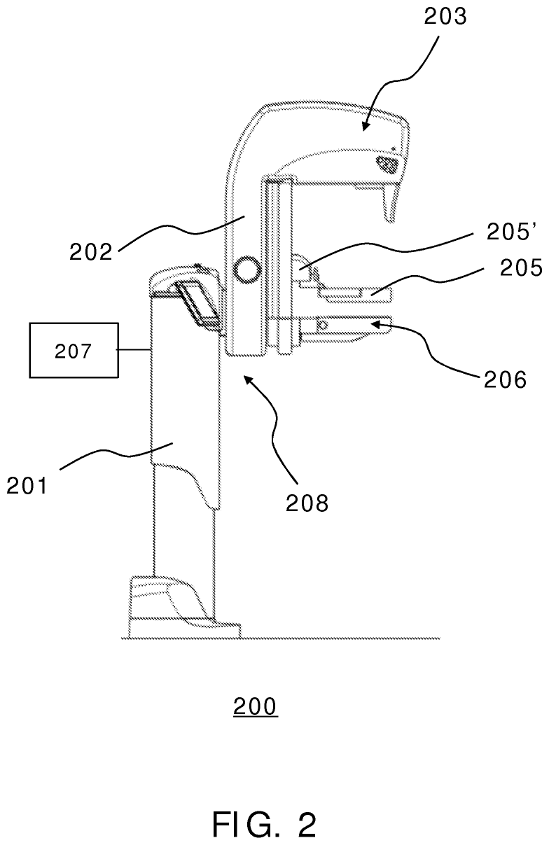

FIG. 2 shows a side view of an x-ray imaging device according to an embodiment of this disclosure. The imaging device 200 comprises a vertically extending frame 201 and an elongated rotatable arm 202 which is operatively connected to the vertically extending frame 201, having a first end and a second end. The rotatable arm 202 comprises a radiation source 203 arranged at a proximity of the first end and an image detector 206 at a proximity of the second end thereof. The imaging device 200 further comprises a compression plate 205 connected to the rotatable arm 202 between the radiation source 203 and the image detector 206, arranged movable alongside the rotatable arm 202 by a motorized mechanism 208, and a control system 207 for operating the imaging device. The control system 207 may further include algorithms for e.g. processing image data and a workstation, which may further comprise a computer, a display, a table, and a radiation shield (not shown in FIG. 2). The imaging device 200 may further comprise a stand, which may be fixed, either resting freely on the floor or bolted to the floor and may further have pedals for controlling the imaging device.

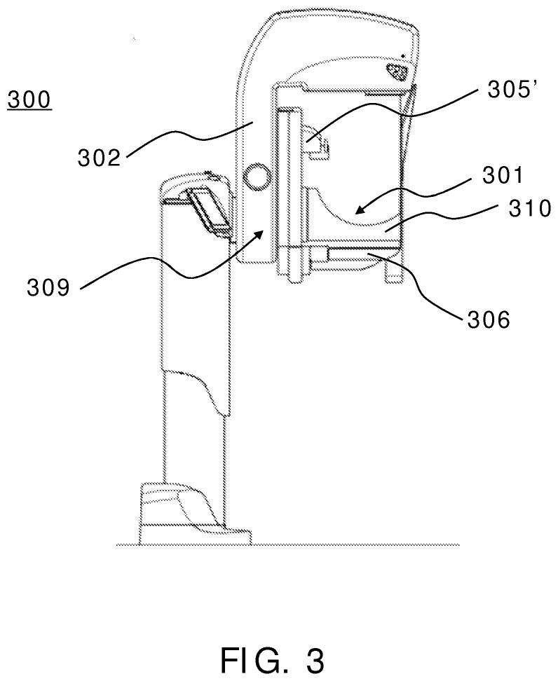

FIG. 3 shows a side view of an x-ray imaging device shown in FIG. 2 when a breast positioning structure is connected therein. For operation in computed tomography mode the compression plate (and a so called bucky) may be removed. In FIG. 3, the connection element 305′ has been moved up towards the radiation source. The image detector 306 may be moved a distance downwards relative to the radiation source 203. The breast positioning structure 310 is arranged above the image detector 306 and connected to the connection structure 309 arranged to the elongated rotatable arm 202. The breast positioning structure 310 comprises for at least part of its length a tubular and open breast positioning surface 301.

In FIG. 3, the breast positioning structure 310 comprises a first component in form of an elongated element extending in a first direction and having at its first end a connecting means 311 compatible with said connection structure 309 and at its second end as integrated thereto a second component of the breast positioning structure 310, said elongated element of the breast positioning structure comprising for at least part of its length a tubular but open breast positioning surface 301, and wherein said second component is a plate-like protective cover 303 comprising a surface extending essentially on a plane in a second direction which is at right angle to the first direction, and wherein the protective cover 303 comprises an opening combing to the breast positioning surface 301.

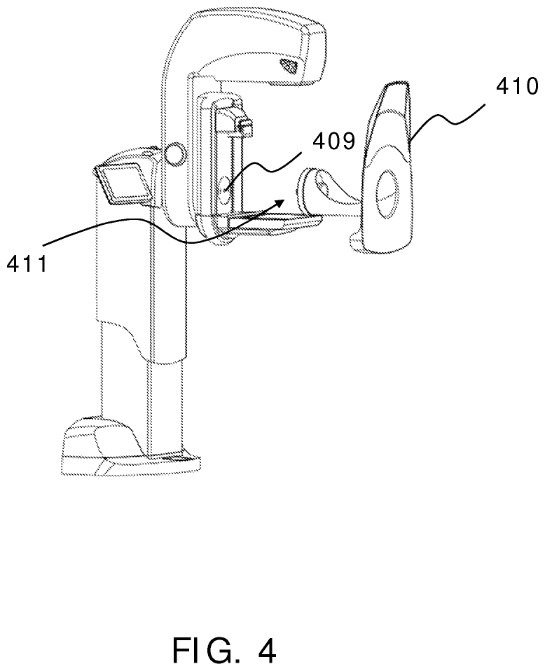

FIG. 4 is an exploded perspective view of an x-ray imaging device illustrated in in FIG. 3 with the breast positioning structure 410 disconnected. The breast positioning structure 410 is shown on the right and the imaging device on the left. The imaging device comprises a connection structure 409 compatible with connection means 411.

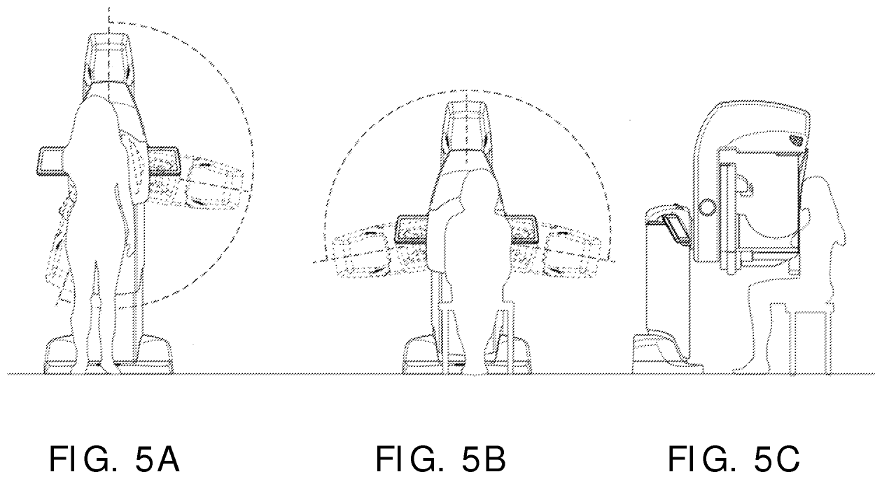

FIG. 5A is an embodiment of the present disclosure in which a patient stands at an imaging device during imaging and the rotatable arm rotates around the patient's breast, on left side, over a scanning angle of about 200 degrees. The dashed line shows the scan angle.

FIG. 5B is an embodiment of the present disclosure in which a patient is in a sitting position at an imaging device during imaging and the rotatable arm rotates around the patient's breast over a scanning angle of about 200 degrees. The dashed line shows the scan angle.

FIG. 5C is a side view of FIG. 5B, in which the patient sits at the imaging device during imaging.

During use of constructions as discussed above, the patient can place the breast to be imaged in the imaging device by herself. For the breast placement, the breast positioning structure may be provided with an opening, which may be of a given size and shape. The breast positioning structure may be open upwards at least partially so that the opening may extend along the breast positioning structure. The purpose is to make it easy for the patient to independently position her breast in the structure without assistance of an outside person, such as an imaging device operator.

In one aspect, an embodiment of the present disclosure provides an imaging device for x-ray imaging a breast of a standing or a sitting patient, which imaging device comprises a vertically extending frame; an elongated rotatable arm having a rotation axis, which rotatable arm is operatively connected to the vertically extending frame. The rotatable arm has a first end and a second end, a radiation source arranged at a proximity of the first end and an image detector at a proximity of the second end thereof. A compression plate may be connected or arranged connectable to the rotatable arm between the radiation source and the image detector, and arranged movable alongside the rotatable arm. The imaging device may comprise a control system.

In an embodiment of the present disclosure, the rotatable arm comprises a connection structure between the first end and second end to removably receive a breast positioning structure, wherein the breast positioning structure comprises a first component in form of an elongated element extending in a first direction and having at its first end a connecting means compatible with said connection structure at the rotatable arm and at its second end, as integrated thereto, a second component of the breast positioning structure generally extending in a second direction perpendicular to the first direction. The elongated element of the breast positioning structure comprises for at least part of its length a tubular but open breast positioning surface. Said second component may be a plate-like protective cover comprising a surface extending substantially on a plane in a second direction which is substantially at right angles to the first direction, and wherein the protective cover comprises an opening combing to the breast positioning surface.

The imaging device as described in the present disclosure provides an integration of a mammography device and a computed tomography device into the same imaging device. The imaging device may therefore be used in different modes of operation and be configured to be selectively used for both traditional 2D mammography and computed tomography. Considering the imaging device in a position where the rotatable arm extends vertically with the first end of it being the upper end, transition from mammography mode to computed tomography mode may be done by removing from the rotatable arm the compression plate possibly used in mammography mode and by connecting to the rotatable arm the breast positioning structure with a protective cover. The rotatable arm may comprise above the image detector a connector structure for the breast positioning structure. In connection with the transition, the image detector may be moved a distance downwards, for example in the range of 1 mm-100 mm, for example 50 mm, 80 mm or 100 mm downwards, as seen from the radiation source.

An imaging volume of the imaging device in the context of the current disclosure may be designed to have a diameter of 200 mm and be dimensioned based on a maximum breast diameter of 190 mm and a determined maximum breast length of 135 mm. When the patient's breast is supported on the breast positioning surface, its shape will change due to the gravity. The deformation spreads the breast while magnitude of the spread depends on the density of the breast tissue. The inner surface of the positioning structure is therefore formed as trough-like or tubular so that it will prevent the breast from spreading in the width direction and spreading outside the imaging volume. The positioning structure is positioned in the imaging device so that its lowest point is 20 mm, for example, above the lowest point of the imaging volume.

In one aspect, an embodiment of the present disclosure provides the breast positioning structure with a protective cover which has “at its lowermost end a portion configured to extend substantially in parallel with the elongated element connecting to the rotatable arm and an uppermost portion, which upper portion is configured to extend in a direction at an angle away from the imaging device.

The role of the protective cover in the breast positioning structure is to adequately protect the patient in a standing or in a sitting position from collisions with a moving rotating arm, and that the patient's anatomy apart from the breast is not exposed to the radiation during imaging operation. The protective cover is adapted to extend from the edges of the first opening along the patient's body over the patient's chest and head, and thereby advantageously to conform to the shape of the patient's body to protect it. The protective cover may have a width which is substantially greater than the width of the patient's breast. The height of the protective cover may be adapted to extend to the proximity of the radiation source. The protective cover can be a separate or an integral part of the positioning structure and made of a material that is rigid and radiolucent, for example made of carbon fiber. The protective cover may be, for example, multi-part. The upper portion of the protective cover may be transparent, e.g., made of polycarbonate while the lower portion may be opaque. The protective cover may be shaped in an angular position relative to the lower portion of the protective cover, e.g., so that the upper portion protrudes at an angle to the patient's face to guiding position of patent's head and body members as desired. In the sitting position, for computed tomography imaging, the protective cover is further designed to be at a sufficient distance from the floor. The distance can be, for example, 750 mm, the value which is based on statistics for average patient sizes when they are in a sitting position. The distance from the edge of the protective cover to the thighs of the seated patient is as small as possible, for example about 160 mm, so that the image detector can rotate around the object to be imaged in the sitting position of the patient. In mammography mode, the minimum size is higher.

During use, the patient can place the breast to be imaged in the imaging device by herself. For the breast placement, the positioning structure may be provided with a second opening, which may have different sizes and shapes. The positioning structure may be open upward at least partially so that the opening may extend along the positioning structure. The purpose is to make it easy for the patient to independently position the breast in the positioning structure without the assistance of an outside person, such as an imaging device operator, and on the other hand, to enable the breast positioning without compression, or flattening. When the second opening is properly sized for the size of the patient's breast, it is easy for the patient to install in place to the breast positioning surface.

The mechanism for moving the image detector in the imaging device has a connection structure, for example a locking means, or a quick-locking means, and provided, for example, with a bearing, and the second end of the positioning structure has a connection means designed to releasable engage each other and configured to allow rotational movement of the positioning structure in the opposite direction to the arm and the image detector, such that the positioning structure remains in place relative to the patient during rotation. The connection of the positioning structure can be implemented, coaxially, for example, in such a way that positioning structure engages an independent rotation mechanism which allows rotation about the same axis as the arm of the imaging device. In addition, the breast positioning surface of the positioning structure is spaced from the image detector, for example 5 mm, 10 mm, 20 mm, 50 mm or 80 mm above the image detector and arranged so that the radiation source and the image detector can rotate freely around the positioning structure in opposite directions while the user's breast is on the breast positioning surface.

In the present disclosure there are two specific modes of operation for the imaging device when a positioning structure is connected to the imaging device. In computed tomography mode, when the patient is in a standing position, the rotatable arm, with the radiation source and image detector, is adapted to be rotated in a scanning trajectory which extends 180 or 200 degrees or more from above the patient's head to the left when the left breast is to be imaged, and 180 or 200 degrees or more from above the patient's head to the right when the right breast is to be imaged. In addition, when the patient is in a sitting position, the rotatable arm, with the radiation source and image detector, is adapted to be rotated in a scanning trajectory of 200 degrees or more, extending 100 degrees or more from above the patient's head to the left and 100 degrees or to the right. The above modes allow the imaging device to image one breast at a time while the patient is sitting and one breast at a time when the patient is standing.

In the following the use of an imaging device according to the present disclosure is described.

When the imaging device is in mammography mode and transits to computed tomography mode, the transition is made by removing the upper compression plate and bucky from the imaging device so that they do not interfere with imaging in computed tomography mode. Thereafter, the assembly formed by the compression mechanism and the image detector in the imaging device is moved by a control system in a motorized manner, for example 80 mm downwards from the radiation source. The connection mechanism the for compression plate may be in a similar way moved upward, when the upper compression plate is removed. After this the above-described positioning structure is connected to the imaging device via the connection structure. The positioning structure is connected such that the protective cover is towards the patient and the connecting means of the positioning structure is towards the imaging device.

In computed tomography mode, the patient to be imaged is positioned in a standing position against the positioning structure so that the patient's breast is positioned through the first opening on the trough-like breast positioning surface. The patient can place the breast to be imaged on the surface by herself because compression is not used. During imaging, the arm and associated radiation source and image detector rotate around the breast to be imaged, exposing, for example, 350 images with a 180 degree or 200-degree scanning angle from above the patient's head to left or right. The positioning structure rotates in the opposite direction to the movement of the arm and thus remains stationary relative to the patient. The imaging trajectory can be defined as symmetrical or asymmetrical. If the patient is in a sitting position, the vertical frame of the imaging device is adjusted so that the lower edge of the protective cover is approximately 160 mm above the patient's thigh when seated.

Embodiments of this disclosure thus include an imaging device for x-ray imaging a breast of a standing or a sitting patient, comprising a vertically extending frame (201), an elongated rotatable arm (202, 302) having a rotation axis, which elongated rotatable arm (202, 302) has a first end and a second end is operatively connected to the elongated rotatable frame (201), the elongated rotatable arm (202, 302) comprising a radiation source (203) arranged at a proximity of the first end and an image detector (206, 306) at a proximity of the second end thereof; a compression plate (205, 305) optionally removably connected to the elongated rotatable arm (202, 302) between the radiation source (203) and the image detector (206, 306) and arranged movable alongside the elongated rotatable arm (202, 302) and a control system (207). The elongated rotatable arm (202, 302) comprises a connection structure (309, 409) between the first end and the second end thereof to removably receive a breast positioning structure (110, 310, 410), wherein the breast positioning structure (110, 310, 410) comprises a first component in form of an elongated support element (112) having a first end and a second end and extending in a first direction and having at its first end a connecting means (111) compatible with said connection structure (309, 409) at said elongated rotatable arm (202, 302), said elongated support element (112) comprising for at least part of its length a tubular breast positioning surface (101, 301), and wherein said breast positioning structure (110, 310, 410) further comprises a second component in form of a plate-like protective cover (103) comprising a surface extending essentially on a plane in a second direction which is generally at right angles to the first direction, and wherein the protective cover (103) comprises an opening (104) at least partly uniting to the breast positioning surface (101, 301).

The elongated support element (112) may be for a part of its length tubular and for another part tubular but open as for its upper part.

The breast positioning structure (110, 310, 410) may be configured to be coaxially rotatable with the elongated rotatable arm (202, 302) in a direction opposite to direction of rotation of the elongated rotatable arm (202, 302).

The image detector (206, 306) may be arranged movable relative to the elongated rotatable arm (202, 302) so as to locate at different distances from the radiation source (203)

The control system may comprise different modes of operation for imaging, to be used in conventional 2D mammography and, on the other hand, in computed tomography.

A computed tomography mode may comprise adjusting distance from the radiation source (203) to the image detector (206, 306) to be in a range of 1-100 mm longer than in a 2D mammography mode.

The elongated rotatable arm (202, 302) may be configured to rotate over an angular range of more than 180 degrees

The elongated support element (112) may be made of carbon fiber.

The protective cover (103) may have a width which is substantially greater than the width of a patient's breast and a height adapted to extend to the proximity of the radiation source.

The breast positioning structure (110, 310, 410) may have a protection part (105) which extends from a lower end of the plate like protection cover (103) in the same direction as the elongated support element (112).

The method may further comprise, when switching from the mammography to the computed tomography mode, adjusting distance between the radiation source (203) and image detector (206, 306).

Embodiments of this disclosure also include a method for x-ray imaging a breast of a standing or a sitting patient with an imaging device (200, 300) which may comprise, when switching from a mammography imaging mode to a computed tomography mode: connecting a positioning structure (110, 310, 410) having a breast positioning surface (101) to the imaging device (200, 300); optionally adjusting a distance between a breast positioning surface (101) and an image detector (206, 306) such that the breast positioning surface (101) is at a desired distance from image detector (206, 306); placing the patient's breast to be imaged on the breast positioning surface (101) so that the breast is on the breast positioning surface (101), and operating the imaging device in the computed tomography mode.

The method may comprise, when switching from the mammography imaging mode to the computed tomography mode, adjusting distance between the radiation source (203) and image detector (206, 306).

When switching from the mammography imaging mode to the computed tomography mode, removing a compression plate (205, 305) of the mammography device from the imaging device (200, 300).

When the positioning structure (110, 310, 410) comprises a protective cover having an opening (104) the method may comprise, prior to initiating imaging in the computed tomography mode, positioning the patient against the protective cover (103) such that the patient's chest is facing the imaging device (200, 300), and the breast to be imaged is positioned on the breast positioning surface (101) so that the breast to be imaged passes through the opening (104).

The protective cover (103) may adjust and guide the patient's body so that, during imaging, the patient's chest is in a substantially vertical position when the patient is against the protective cover (103).

In computed tomography mode, when the patient is in a standing position, the rotatable arm (202, 302) may be rotated in a scanning trajectory which extends about 200 degrees or more from the vertical position of the elongated rotatable arm to the left, when the left breast is to be imaged, and about 200 degrees or more from the vertical position of the elongated rotatable arm to the right, when the right breast is to be imaged.

In computed tomography mode, further, when the patient is in a sitting position, the rotatable arm (202, 302) may be rotated in a scanning trajectory of about 200 degrees or more, extending about 100 degrees from the vertical position of the elongated rotatable arm to the left and about 100 degrees to the right.

During a computed tomography exposure, the positioning structure may be rotated (110, 310, 410) about the same rotation axis in a direction opposite to a direction of rotation of the rotatable arm (102).

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. An imaging device for x-ray imaging a breast of a standing or a sitting patient, the imaging device comprising:
a vertically extending frame;
an elongated rotatable arm having a rotation axis, which elongated rotatable arm has a first end and a second end is operatively connected to the vertically extending frame, the elongated rotatable arm comprising a radiation source arranged at a proximity of the first end and an image detector at a proximity of the second end thereof;
a compression plate optionally removably connected to the elongated rotatable arm between the radiation source and the image detector; and arranged movable alongside the elongated rotatable arm;
a control system, characterized in that the elongated rotatable arm comprises a connection structure between the first end and the second end thereof to removably receive a breast positioning structure, wherein the breast positioning structure comprises a first component in form of an elongated support element having a first end and a second end and extending in a first direction and having at its first end a connecting means compatible with said connection structure at said elongated rotatable arm, said elongated support element comprising for at least part of its length a tubular breast positioning surface, and
wherein said breast positioning structure further comprises a second component in form of a plate-like protective cover comprising a surface extending essentially on a plane in a second direction which is generally at right angles to the first direction, and wherein the protective cover comprises an opening at least partly uniting to the breast positioning surface.

2. The imaging device according to claim 1, characterized in that the elongated support element is for a part of its length tubular and for another part tubular but open as for its upper part.

3. The imaging device according to claim 1, characterized in that the breast positioning structure is configured to be coaxially rotatable with the elongated rotatable arm in a direction opposite to direction of rotation of the elongated rotatable arm.

4. The imaging device according to claim 1, characterized in that the image detector is arranged movable relative to the elongated rotatable arm so as to locate at different distances from the radiation source.

5. The imaging device according to claim 1, characterized in that the control system comprises different modes of operation for imaging, to be used in conventional 2D mammography and, on the other hand, in computed tomography.

6. The imaging device according to claim 5, characterized in that a computed tomography mode comprises adjusting distance from the radiation source to the image detector to be in a range of 1 mm longer than in a 2D mammography mode.

7. The imaging device according to claim 1, characterized in that the elongated rotatable arm is configured to rotate over an angular range of more than 180 degrees

8. The imaging device according to claim 1, characterized in that the elongated support element is made of carbon fiber.

9. The imaging device according to claim 1, characterized in that the protective cover has a width which is substantially greater than the width of a patient's breast and a height adapted to extend to the proximity of the radiation source.

10. The imaging device according to claim 1, characterized in that the breast positioning structure has a protection part which extends from a lower end of the plate like protection cover in the same direction as the elongated support element.

11. A method for x-ray imaging a breast of a standing or a sitting patient with an imaging device, characterized in that the method comprises, when switching from a mammography imaging mode to a computed tomography mode:
connecting a positioning structure having a breast positioning surface to the imaging device;
optionally adjusting a distance between a breast positioning surface and an image detector such that the breast positioning surface is at a desired distance from image detector;
placing the patient's breast to be imaged on the breast positioning surface so that the breast is on the breast positioning surface,
operating the imaging device in the computed tomography mode.

12. A method according to claim 11, characterized in that, the method comprises
when switching from the mammography imaging mode to the computed tomography mode, adjusting distance between the radiation source and image detector.

13. A method according to claim 11, characterized in that, the method comprises
when switching from the mammography imaging mode to the computed tomography mode, removing a compression plate of the mammography device from the imaging device.

14. A method according to claim 11, characterized in that the positioning structure further comprises a protective cover having an opening and that the method comprises, prior to initiating imaging in the computed tomography mode,
positioning the patient against the protective cover such that the patient's chest is facing the imaging device, and the breast to be imaged is positioned on the breast positioning surface so that the breast to be imaged passes through the opening.

15. A method according to claim 11, characterized in that, the method comprises
arranging the protective cover to adjust and guide the patient's body so that, during imaging, the patient's chest is in a substantially vertical position when the patient is against the protective cover.

16. A method according to claim 11, characterized in that, the method comprises
in computed tomography mode, when the patient is in a standing position, rotating the rotatable arm in a scanning trajectory which extends about 200 degrees or more from the vertical position of the elongated rotatable arm to the left, when the left breast is to be imaged, and about 200 degrees or more from the vertical position of the elongated rotatable arm to the right, when the right breast is to be imaged.

17. A method according to claim 11, characterized in that, the method comprises
incorporated tomography mode, when the patient is in a sitting position, rotating the rotatable arm in a scanning trajectory of about 200 degrees or more, extending about 100 degrees from the vertical position of the elongated rotatable arm to the left and about 100 degrees to the right.

18. A method according to claim 11, characterized in that, the imaging device comprises a rotatable arm and that the method comprises,
rotating during a computed tomography exposure the positioning structure about the same rotation axis in a direction opposite to a direction of rotation of the rotatable arm.
